(19) United States
(12) Patent Application Publication
(10) Pub. No.: US 2018/0355304 A1
(43) Pub. Date: Dec. 13, 2018

(54) CHEMICALLY DEFINED MEDIA FOR THE GROWTH OR DETECTION OF MICROORGANISMS

(71) Applicants: Merck Patent GmbH, Darmstadt (DE); Universite de Strasbourg, Strasbourg Cedex (FR); Centre National de la Recherche Scientifique, Paris Cedex 16 (FR)

(72) Inventors: Renaud CHOLLET, Selestat (FR); Antoine GILLMANN, Strasbourg (FR); Didier LIEVREMONT, Benfeld (DE); Marie-Claire LETT, Schiltigheim (FR)

(73) Assignees: Merck Patent GmbH, Darmstadt (DE); Universite de Strasbourg, Strasbourg Cedex (FR); Centre National de la Recherche Scientifique, Paris Cedex 16 (FR)

(21) Appl. No.: 15/780,669

(22) PCT Filed: Nov. 8, 2016
(86) PCT No.: PCT/EP2016/001845

§ 371 Date: Jun. 1, 2018

(30) Foreign Application Priority Data
Dec. 3, 2015 (EP) 15290300.1

(51) Int. Cl. C12N 1/38; C12N 1/14; C12N 1/20

(52) U.S. Cl. CPC C12N 1/38; C12Q 1/045; C12N 1/20; C12N 1/14

(57) ABSTRACT

The present invention relates to chemically defined culture medium comprising at least one osmoprotectant compound and at least one fatty acid for the detection of a broad range of microorganisms.

The present invention relates to chemically defined culture medium comprising at least one osmoprotectant compound and at least one fatty acid for the detection of a broad range of microorganisms.

General purpose culture media named complex media have been available for the growth and culture of bacteria, yeast and molds since the nineteenth century. The process for manufacturing these media and the main components used has surprisingly changed very little in its fundamental nature since this time. The main basis of these microbiological media are the peptones which they contain.

The peptones present are designed to be present in combinations and at concentrations which allow growth of a very broad variety of bacteria, yeast and molds. Thus, mixtures of these peptones are a biochemically rich and well balanced nutrient source and are usually complemented by the addition of salts, buffers and other raw materials necessary to optimize speedy growth. In addition, the solid form of the medium is generally well soluble in water. After dissolving, the medium is sterilisable and can be provided in a suitably convenient container to prevent contamination but at the same time allow in oxygen, if this is required, after inoculation with prokaryotic and/or eukaryotic cells. Subsequently, the container with medium and cells is incubated at a suitable temperature to allow cell growth.

However, only a careful selection of peptones types by highly experienced individuals familiar with the art will yield an adequate medium with a nutritional basis broad enough and sufficiently balanced in its biochemical composition to enable the cultivation of a very wide range of prokaryotes as well as eukaryotes. Peptone quality varies between peptone types, between different manufacturers of the same peptone, varies between different grades from the same manufacturer and even varies significantly between batches of the same grade from a single manufacturer. However, the biochemical nature of this varying quality is poorly understood, more especially so at the batch-to-batch variation level. However, the biochemical variation is certainly in part due to the varying quality of the natural raw materials used. Thus, not all batches of peptones are always suitable for use in particular media recipes, due to nutritional deficiencies or nutritional imbalances in such raw materials and hence each batch must be carefully selected to enable growth of a very wide range of prokaryotes and eukaryotes, i.e. support acceptable growth of a wide variety of bacteria, yeast and molds.

The precise composition of the peptones is not known. This means the critical nutritional factors provided by such natural raw materials resulting in cell growth promotion is understood up to a point but not well. Poorly understood and equally difficult to control are the negative physico-chemical interactions of such raw materials within a medium which cause it to precipitate, for example. The complexity of the problem increases when one peptone is mixed with other peptone types in addition to the salts, buffers and other components typically present in a traditional medium. The manufacture of well-balanced, general growth media is a matter of trial, error and long experience. Batch-to-batch variation of the individual peptones added must be adequately controlled such that batches unsuitable to yield the full range of desired cellular growth of the test prokaryotic (bacterial) and eukaryotic (yeast and fungal) strains, whether alone or in combination with other peptone types, can be eliminated from the production processes to manufacture such media.

Even after these measures such general media for the growth of a very wide range of cell strains are not necessarily sufficiently biochemically balanced or nutritious to grow all the bacteria, yeast and fungal strains tested in a broad test panel typically used to confirm media suitability for environmental monitoring applications. On the one side certain cell types are very sensitive to the correct balance of biochemicals offered in the medium. On the other hand, and in addition, particularly very fastidious cells need extra supplements like fresh blood or blood extracts to boost their growth performance. Even these media do not have a sufficient nutrient composition for certain very highly fastidious cell types and need a further addition of supplement(s) to support growth or to speed up growth in order to make them effective for their use for a broad range of prokaryotic and eukaryotic cells typically found in environmental samples.

Lately, chemically defined cell culture media have been developed. However, to date, producers of chemically defined media for the culture of microorganisms have concentrated on supporting the growth of either prokaryotes (e.g. bacteria) or eukaryotes (e.g. yeasts or fungi or insect or mammalian cells).

WO 2007/135385 for example discloses a chemically defined medium for the growth of bacteria, especially Neisseria species.

GB 2464203 discloses a chemically defined medium for the enumeration of Campylobacter.

The aim of the present invention is thus to provide cell culture media based on chemically defined raw materials that offer a foundation for growing a very broad range of microorganisms, such as prokaryotic as well as eukaryotic species comparable to the well-known peptone and/or extract-based media for example, covering the needs of growing the prokaryotic and eukaryotic cells typically found in various samples, such as environmental samples, samples from food and beverage industry, pharmaceutical samples or clinical samples.

It has been found that a general, non-specific, chemically defined growth medium capable of supporting growth of a very broad range of prokaryotes and eukaryotes typically found in various samples, with growth potential essentially the same as traditional peptone-based media, can be provided. The medium not only fulfils an appropriate biochemical balance to grow the majority of typical isolates, both prokaryote and eukaryote but, at the same time fulfils the requirements for supporting the growth of fastidious cells both prokaryotes and eukaryotes. Concomitantly, such media also typically enable a much speedier growth of yeast and molds than traditional peptone-based complex media at defined incubation conditions.

Consequently, the present invention is directed to a chemically defined culture medium comprising at least one osmoprotectant compound and at least one fatty acid.

It was found that the combination of at least one osmoprotectant compound and at least one fatty acid allows to generate a universal chemically-defined and fully synthetic culture medium promoting the growth of diverse microorganisms.

An osmoprotectant compound (or compatible solute) is a small molecule that acts as osmolyte and helps organisms survive extreme osmotic stress. These molecules accumulate in the cells and balance the osmotic difference between the cell's surroundings and the cytosol. Typical osmoprotectant categories are for example amino acids, sugars or polycations (e.g. proline, choline, saccharose, trehalose, spermidine . . . ). Preferred categories are amino acids or peptides.

In a preferred embodiment the osmoprotectant compound is selected from the group consisting of betaine (trimethylglycine), ectoine, proline, choline, saccharose, trehalose and spermidine. In a particularly preferred embodiment the osmoprotectant is selected from the group consisting of betaine (trimethylglycin) and ectoine. Even more preferably, the osmoprotectant compound is betaine.

Typically, the concentration of the osmoprotectant compound in the culture medium is in the range of 10 to 360 mg/l of culture medium. In a preferred embodiment the concentration of the osmoprotectant compound in the culture medium is in the range of 20 to 160 mg/l of culture medium. In a more preferred embodiment the concentration of the osmoprotectant compound in the culture medium is in the range of 80 to 120 mg/l of culture medium. Even more preferably, the concentration of the osmoprotectant compound is 100 mg/l of culture medium.

A fatty acid is a carboxylic acid with a long aliphatic tail (chain) which is either saturated or unsaturated. The fatty acids to be used according to the present invention can either have an even or an uneven number of carbon atoms and can be saturated or unsaturated. The number of carbon atoms is typically between 4 and 28. Preferably, the number of carbon atoms is between 6 and 22, more preferably between 8 and 20. Unsaturated fatty acids have at least one double bond, but can have also more double bonds, e.g. 2, 3, 4, 5 or 6. Examples of fatty acids to be used according to the present invention are myristoleic acid, oleic acid, linoleic acid, stearic acid, palmitic acid, arachidic acid or sapienic acid. In a preferred embodiment the fatty acid is selected from the group consisting of myristoleic acid, oleic acid, linoleic acid and stearic acid. More preferably, the fatty acid is oleic acid.

Typically, the concentration of the fatty acid in the culture medium is in the range of 5 to 40 mg/l of culture medium. Preferably, the concentration is in the range of 10 to 30 mg/l, more preferably 20 mg/l of culture medium.

A cell culture is any setup in which cells are cultured.

A cell culture can be performed in any container suitable for the culture of cells, such as a petri dish, contact plate, bottle, tube, well, vessel, bag, flask or tank. Typically the container is sterilized prior to use. Incubation is typically performed under suitable conditions such as suitable temperature, osmolarity, aeration, agitation, etc. A person skilled in the art is aware of suitable incubation conditions for supporting or maintaining the growth/culturing of cells.

A cell culture medium (synonymously used: culture medium) according to the present invention is any mixture of components which maintains and/or supports the in vitro growth of cells and/or supports a particular physiological state. It is a chemically defined medium. The cell culture medium can comprise all components necessary to maintain and/or support the in vitro growth of cells or be used for the addition of selected components in combination with further components that are added separately. Preferably the cell culture medium comprises all components necessary to maintain and/or support the in vitro growth of cells.

The cell culture media according to the present invention are designed to be suitable to grow or maintain/support the growth many different kinds of organism, e.g. prokaryotic cells like bacterial cells or eukaryotic cells like yeast, fungi, algae, plant, insect or mammalian cells or archaea. Preferably, they maintain/support the growth of prokaryotic cells and eukaryotic cells.

Examples of cells of which growth is maintained/supported by the media according to the present invention are:
bacteria:
Achromobacter sp. wild type
Acinetobacter lwoffii ATCC® 17925™
Bacillus clausii ATCC® 700160
Bacillus halodurans wild type
Bacillus okuhidensis wild type
Bacillus pumilus wild type
Bacillus subtilis ATCC® 6633™
Bacillus subtilis wild type
Burkholderia sp. wild type
Clostridium sporogenes ATCC® 11437™
Clostridium sporogenes ATCC® 19404™
Corynebacterium tuberculostearicum wild type
Escherichia coli ATCC® 25922™
Escherichia coli ATCC® 8739™
Kocuria rhizophila ATCC® 9341™
Leifsonia sp. wild type
Methylobacterium extorquens ATCC® 43645™
Methylobacterium extorquens NBRC 15911
Methylobacterium fujisawaense wild Type
Methylobacterium mesophilicum ATCC® 29983™
Methylobacterium ssp. wild type
Micrococcus luteus ATCC® 10240™
Micrococcus lylae wild type
Paenibacillus lautus wild type
Pantoea sp. wild type
Propionibacterium acnes ATCC® 6919™
Pseudomonas aeruginosa ATCC® 9027™
Ralstonia pickettii ATCC® 27511™
Ralstonia pickettii wild type
Salmonella typhimurium ATCC® 14028™
Serratia marcescens wild type
Sphingomonas parapaucimobilis wild type
Sphingomonas paucimobilis ATCC® 29837™
Sphingomonas paucimobilis wild type
Staphylococcus aureus ATCC® 25923™
Staphylococcus aureus ATCC® 6538™
Staphylococcus epidermidis ATCC® 12228™
Staphylococcus epidermidis wild type
Staphylococcus hominis ATCC® 27844™
Stenotrophomonas maltophilia ATCC® 13637™
Streptococcus pyogenes ATCC® 12344™
Streptococcus pyogenes ATCC® 21059™
   yeasts:
Candida albicans ATCC® 10231™
Debaryomyces hansenii DSM 3428
Exophiala sp. wild type
   molds:
Aspergillus brasiliensis ATCC® 16404™
Aspergillus brasiliensis wild type
Aspergillus sydowii DSM 63373
Penicillium commune ATCC® 10428™

For example, the chemically defined culture medium according to the present invention may support the growth of Gram-positive microorganisms and Gram-negative microorganisms, such as human skin contaminants, water contaminants, yeast and mold, e.g. Bacillus subtilis, Clostridium sporogenes, Propionibacterium acnes, Staphylococcus aureus, Aspergillus brasiliensis, Candida albicans, Escherichia coli, Methylobacterium extorquens, Methylobacterium fujisawaense, Methylobacterium mesophilicum, Pseudomonas aeruginosa, Paenibacillus lautus, Ralstonia pickettii, Staphylococcus epidermidis.

Chemically defined cell culture media are cell culture media comprising of chemically well characterized ‘defined’ raw materials. This means that the chemical composition of all the chemicals used in the media is known. The chemically defined media do not comprise of chemically ill-defined yeast, animal or plant tissues; they do not comprise peptones, feeder cells, serum, extracts or digests or other components which may contribute chemically poorly defined proteins and/or peptides and/or hydrolysates to the media. Chemically undefined or poorly defined chemical components are those whose chemical composition and structure is not well known, are present in poorly defined and varying composition or could only be defined with enormous experimental effort—comparable to the evaluation of the chemical composition and structure of a protein-digest from albumin or casein. The chemically defined medium may comprise proteins or peptides which are chemically defined.

A powdered cell culture medium or a dry powder medium is a cell culture medium typically resulting from a milling process or a lyophilisation process. That means the powdered cell culture medium is typically a finely granular, particulate medium—not a liquid medium. The term “dry powder” may be used interchangeably with the term “powder;” however, “dry powder” as used herein simply refers to the gross appearance of the granulated material and is not intended to mean that the material is completely free of complexed or agglomerated solvent unless otherwise indicated. A powdered cell culture medium can also be a granulated cell culture medium, e.g. dry granulated by roller compaction.

The media of the present invention for supporting the general growth of prokaryotes and eukaryotes show comparable (visible) growth characteristics to standard media used for this purpose, thus they typically
a. contain a balance of biochemicals to promote cell growth of the majority of cells which tend to be detected
   i) contain sufficient specific biochemicals to supply the needs of a very wide range of prokaryotes and eukaryotes but at the same time
   ii) not contain concentrations of specific biochemical which may be so high as to significantly inhibit the growth of certain sensitive cell strains
b. contain a rich nutrient base suitable to bridge the auxotrophic gaps present in many prokaryotic and eukaryotic species
c. contain certain complex biochemicals able to feed cells such that growth is not delayed unnecessarily by extensive de novo enzyme synthesis leading to a
   i) significantly extended lag phase or
   ii) death of the cells since they are not able to recover sufficient metabolic activity to overcome cellular damage and/or cellular anabolic activity

A cell culture medium which comprises all components necessary to maintain and/or support the in vitro growth of cells typically comprises at least one or more saccharide components, one or more amino acids, one or more vitamins or vitamin precursors, one or more salts, one or more buffer components, one or more co-factors and one or more nucleic acid components (nitrogenous bases).

It may also comprise recombinant proteins, e.g. rinsulin, rBSA, rTransferrin, rCytokines etc. Preferably, the medium does not comprise recombinant proteins.

The media may also comprise sodium pyruvate, fatty acids and/or fatty acid derivatives and/or pluronic product components (block copolymers based on ethylene oxide and propylene oxide) in particular Poloxamer 188 sometimes called Pluronic F 68 or Kolliphor P 188 or Lutrol F 68 and/or surface active components such as chemically prepared non-ionic surfactants. One example of a suitable non-ionic surfactant are difunctional block copolymer surfactants terminating in primary hydroxyl groups also called poloxamers, e.g. available under the trade name Pluronic® from BASF, Germany. Such pluronic product components are in the following just called pluronic. Chelators, hormones and/or growth factors may also be added.

Other components it may comprise are the pure compounds, salts, conjugates, and/or derivatives of lactic acid, thioglycollic acid, thiosulphates, tetrathionate, diaminobutane, myo-inositol, phosphatidylcholine (lecithin), sphingomyelin, iron containing compounds (including iron sulphur clusters), uric acid, carbamoyl phosphate, succinic acid, orotic acid, phosphatidic acid, putrescine, triglycerides, steroids (including cholesterol), metallothionine, oxygen, glycerol, urea, alpha-ketoglutarate, ammonia, glycerophosphates, starch, glycogen, glyoxylate, isoprenoids, methanol, ethanol, propanol, butanol, acetone, lipids (including those in micelles), tributyrin, butyrin, cholic acid, desoxycholic acid, polyphosphate, acetate, tartrate, malate, oxalate and/or acetone.

Saccharide components are all mono- or di-saccharides, like glucose, galactose, ribose or fructose (examples of monosaccharides) or sucrose, lactose or maltose (examples of disaccharides) or derivatives thereof like sugar alcohols. Saccharide components may also be oligo- or polysaccharides.

Examples of amino acids according to the invention are the proteinogenic amino acids, especially the essential amino acids, leucine, isoleucine, lysine, methionine, phenylalanine, threonine, tryptophan and valine, as well as the non-proteinogenic amino acids such as D-amino acids.

Tyrosine means L- or D-tyrosine, preferably L-tyrosine.

Cysteine means L- or D-cysteine, preferably L-cysteine.

Amino acid precursors and analogues are also included.

Examples of vitamins are Vitamin A (Retinol, retinal, various retinoids, and four carotenoids), Vitamin B₁ (Thiamine), Vitamin B₂ (Riboflavin), Vitamin B₃ (Niacin, niacinamide), Vitamin B₅ (Pantothenic acid), Vitamin B₆ (Pyridoxine, pyridoxamine, pyridoxal), Vitamin B₇ (Biotin), Vitamin B₉ (Folic acid, folinic acid), Vitamin B₁₂ (Cyanocobalamin, hydroxycobalamin, methylcobalamin), Vitamin C (Ascorbic acid) (including phosphates of ascorbic acid), Vitamin D (Ergocalciferol, cholecalciferol), Vitamin E (Tocopherols, tocotrienols) and Vitamin K (phylloquinone, menaquinones). Vitamin precursors and analogues are also included.

Examples of salts are components comprising inorganic ions such as bicarbonate, calcium, chloride, magnesium, phosphate, potassium and sodium or trace elements such as Co, Cu, F, Fe, Mn, Mo, Ni, Se, Si, Ni, Bi, V and Zn. Examples are copper(II) sulphate pentahydrate (CuSO₄.5 H₂O), sodium chloride (NaCl), calcium chloride (CaCl₂.2 H₂O), potassium chloride (KCl), iron(II)sulphate, sodium phosphate monobasic anhydrous (NaH₂PO₄), magnesium sulphate anhydrous (MgSO₄), sodium phosphate dibasic anhydrous (Na₂HPO₄), magnesium chloride hexahydrate (MgCl₂.6 H₂O), zinc sulphate heptahydrate (ZnSO₄.7H₂O).

Examples of buffers are carbonate, citrate, phosphate, HEPES, PIPES, ACES, BES, TES, MOPS and TRIS.

Examples of cofactors are compounds, salts, complexes and/or derivatives of thiamine derivatives, biotin, vitamin C, calciferol, choline, NAD/NADP (reduced and/or oxidized), cobalamin, vitamin B12, flavin mononucleotide and derivatives, flavin adenine dinucleotide, glutathione (reduced and/or oxidized and/or as dimer), haeme, haemin, haemoglobin, ferritin, nucleotide phophates and/or derivatives (e.g. adenosine phosphates), coenzyme F420, s-adenosyl methionine, coenzyme B, coenzyme M, coenzyme Q, acetyl Co-A, molybdopterin, pyrroloquinoline quinone, tetrahydrobiopterin.

Nucleic acid components, according to the present invention, are the nucleobases, like cytosine, guanine, adenine, thymine, uracil, xanthine and/or hypoxanthine, the nucleosides like cytidine, uridine, adenosine, xanthosine, inosine, guanosine and thymidine, and the nucleotides such as adenosine monophosphate or adenosine diphosphate or adenosine triphosphate, including but not limited to the deoxy- and/or phosphate derivatives and/or dimers, trimers and/or polymers thereof, like RNA and/or DNA.

Compounds may be added which improve the physico-chemical properties, like but not limited to, increasing clarity and/or solubility of the media and/or one or more of its components, without negatively affecting the cell growth properties at the concentrations used, such as chelating agents (e.g. EDTA), antioxidants, detergents, surfactants, emulsifiers (like polysorbate 80), neutralising agents, (like polysorbate 80), micelle forming agents, micelle inhibiting agents and/or polypropylene glycol, polyethylene alcohol and/or carboxymethylcellulose.

The medium typically contains carbohydrates such as sugars and/or sugar mixtures and/or sugar dimers and/or sugar polymers and/or their derivatives. Typically, glucose is one of the main carbohydrate sugar components. Glucose is usually included in the medium at a concentration of 0.001 mM to 250 mM in the aqueous medium solution, more preferably 1 mM to 100 mM, even more preferably 1 mM to 10 mM, most preferably 5 mM.

Typically, the medium comprises each amino acid in a range from 10 mg to 3 g per liter, preferably in a range from 40 mg to 1 g per liter.

The medium typically comprises vitamins. A typical amount of a vitamin in the medium is in the range of 5 μg to 10 mg per liter, preferably in the range of 50 μg to 6 mg per liter.

Typically, the medium comprises salts. The amount of one species of salt is typically in the range of 2 μg to 10 mg per liter, preferably in the range of 10 μg to 7 mg per liter, more preferably 1.7 mg per liter up to 6.6 mg per l. Specific salts may also be present in much higher amounts; the concentration of NaCl can for example be up to 5 g per liter.

The typical amount of a nucleic acid comprised in the medium is in the range of 0.5 to 10 mg per liter, preferably in the range of 1 to 5 mg per liter.

The medium typically contains all the proteogenic amino acids (and/or their derivatives and/or their conjugates and/or dimers (pure and/or mixed) thereof). It must be noted that the concentrations in the solid medium may differ significantly to those after dissolution in water since certain amino acids react in the aqueous medium to form products which then indirectly contain the amino acids by which the pure amino acid in solution is thereby depleted. This process may also occur to other easily reactive constituents, for example, but not limited to vitamin C and/or indeed the amino acids may react with each other or with themselves. This process may be an oxidative process dependent on oxygen concentrations and the presence of trace and/or ultra trace elements, in particular the transition metal ions like Cu(II) and/or Fe(III) added directly as components and/or present as a contaminant.

Further components which may be comprised in the medium according to the present invention are:
Ammoniumbicarbonate
Colour indicators
Fluorogenic substrates

The chemically defined culture medium according to the present invention can further comprise at least one fluorogenic substrate. Fluorogenic substrates are complex molecules which, on contact with enzymes synthesized by microorganisms, are cleaved and become fluorescent. The fluorescence emitted is detectable with a spectrophotometer by illuminating the growth medium using radiation in the UV or visible spectrum. Examples of fluorogenic substrates are fluorescein derivatives (fluorescein derivatives, methylumbelliferone derivatives or the Aldols™ (developed by the company Biosynth).

A typically suitable cell culture medium has a typical composition of 2 to 50 g/L, more preferably 5 to 30 g/L. Such a medium with a gelling agent has typically an additional weight due to the gelling agent of between 1 and 50 g/L, more preferably between 2 and 30 g/L.

The osmolality of the medium is typically between 50 mOsm and 1000 mOsm, more preferably between 150 mOsm and 500 mOsm.

The cell culture medium can be a dry powder medium, a liquid medium or a semi-solid medium. In case of a semi-solid medium, the medium comprises beside the chemically defined components a gelling agent. An example of a suitable gelling agent is agarose. In a preferred embodiment the culture medium is a liquid medium.

The present invention is further directed to a method of preparing a culture medium as defined above comprising a step of combining and mixing the components.

Powdered cell culture media are preferably produced by mixing all components and milling them. The mixing of the components is known to a person skilled in the art of producing dry powdered cell culture media by milling. Preferably, all components are thoroughly mixed so that all parts of the mixture have nearly the same composition. The higher the uniformity of the composition, the better the quality of the resulting medium with respect to homogenous cell growth.

The milling can be performed with any type of mill suitable for producing powdered cell culture media. Typical examples are ball mills, pin mills, fitz mills or jet mills. Preferred is a pin mill, a fitz mill or a jet mill, very preferred is a pin mill.

A person skilled in the art knows how to run such mills.

A large scale equipment mill with a disc diameter of about 40 cm is e.g. typically run at 1-6500 revolutions per minute in case of a pin mill, preferred are 1-3000 revolutions per minute.

The milling can be done under standard milling conditions resulting in powders with particle sizes between 10 and 300 μm, most preferably between 25 and 100 μm.

Preferably, all components of the mixture which is subjected to milling are dry. This means, if they comprise water, they do only comprise water of crystallization but not more than 10%, preferably not more than 5% most preferred not more than 2% by weight of unbound or uncoordinated water molecules.

In a preferred embodiment, the milling is performed in an inert atmosphere. Preferred inert protective gas is nitrogen.

In another preferred embodiment, all components of the mixture are frozen prior to milling. The freezing of the ingredients prior to the milling can be done by any means that ensures a cooling of the ingredients to a temperature below 0° C. and most preferably below −20° C. In a preferred embodiment the freezing is done with liquid nitrogen. This means the ingredients are treated with liquid nitrogen, for example by pouring liquid nitrogen into the container in which the ingredients are stored prior to introduction into the mill. In a preferred embodiment, the container is a feeder. If the container is a feeder the liquid nitrogen is preferably introduced at the side or close to the side of the feeder at which the ingredients are introduced.

Typically the ingredients are treated with the liquid nitrogen over 2 to 20 seconds.

Preferably the cooling of the ingredients is done in a way that all ingredients that enter into the mill are at a temperature below 0° C., most preferred below −20° C.

In a preferred embodiment, all ingredients are put in a container from which the mixture is transferred in a feeder, most preferred in a metering screw feeder. In the feeder the ingredients are sometimes further mixed—depending on the type of feeder—and additionally cooled. The frozen mixture is then transferred from the feeder to the mill so that the mixture which is milled in the mill preferably still has a temperature below 0° C., more preferred below −20° C.

Typically the blending time, that means the residence time of the mixture of ingredients in the feeder is more than one minute, preferably between 15 and 60 minutes.

A metering screw feeder, also called dosage snail, is typically run at a speed of 10 to 200 revolutions per minute, preferably it is run at 40 to 60 revolutions per minute.

Typically, the temperature of the mill is kept between −50 and +30° C. In a preferred embodiment, the temperature is kept around 10° C.

The oxygen level during milling preferably is below 10% (v/v).

The process can be run e.g. batch-wise or continuously. In a preferred embodiment the process according to the present invention is done continuously by, over a certain time, permanently filling the mixture of ingredients into a feeder for cooling and permanently filling cooled mixture from the feeder into the mill.

Dry powder cell culture media can also be used as compactates to facilitate handling. Typically compacted media have good dissolving properties and are easier to handle due to reduced dust formation.

The powder media are preferably compacted in a roll press.

A roll press, also called roller compactor, is known to a person skilled in the art. Typically a roll press comprises two counter-rotating rolls which are located at a small distance of about 0.5 to 3 mm, preferably 1 to 2 mm next to each other. Suitable roll presses typically have rolls with a widths between 10 and 50 cm resulting in the gap between the roll having a length between 10 and 50 cm. Nevertheless, the size of the rolls and thus the length of the gap between the rolls can vary depending on the size of the roll press. In a preferred embodiment, the gap has a length between 10 and 15 cm.

The mixed powder material is drawn in between the counter-rotating rolls and compacted in the gap between the rolls. The distance between the rolls and their surface structure have influence on the final size and structure of the resulting granulated particles.

The surface of the rolls is preferably riffled. The riffles help to make the powder stick to the roll and to pull it through the press.

The press capacity of the roll press typically is between 20 and 150 kN/cm roll width, preferably the rolls are pressed together with a force between 30 and 80 kN, most preferred between 40 and 60 kN.

If the components of the cell culture medium are very sensitive, the compacting procedure can be performed under an inert protective gas atmosphere. In addition, the rolls of the roll press are usually cooled to maintain a constant temperature since often some components are heat-sensitive and would not stand the slightly enlarged temperature which might occur due to compaction.

In a preferred embodiment, the compacted cell culture medium which is set free from the roll press is directly sized. This can for example be done by sieving, e.g. with one or more vibrating sieves. The diameter of the holes in the sieve depends on the size of the granules to be collected. For the process according to the present invention, a typical diameter is between 0.5 to 5 mm, preferably around 1 to 3 mm. Especially if the dry granulation in the roll press results in larger compacts or flakes, preferably, a sieve mill is used for granulating the compacts or flakes to granules of suitable size. One suitable sieve mill is the oscillating sieve mill, type FC 200, Bepex GmbH, with a sieve size between 1 and 3 mm.

Roll presses which are suitable for the process according to the present invention, can for example be purchased from Alexanderwerk, Sahut Coreur, Hosokawa or Fitzpatrick Company.

As already explained, the size of the particles of the dry granulated cell culture medium depends on the way the compacted medium which comes out of the roll press is treated. If the medium is directly collected from the roll press, it typically comprises larger compacts or flakes. If the medium is sieved, the particle size is determined by the size of the sieve. But also further handling like packaging typically influences the mean particle size as some particles of the granulated cell culture media might break into pieces.

In a preferred embodiment, after compaction and sieving, more than 80% of the particles of the granulated cell culture medium have a size larger than 0.5 mm.

For use of the milled and/or compacted powdered media a solvent, preferably water (most particularly distilled and/or deionized water or purified water or water for injection) or an aqueous buffer is added to the media and the components are mixed until the medium is totally dissolved in the solvent.

The solvent may also comprise saline, soluble acid or base ions providing a suitable pH range (typically in the range between pH 1.0 and pH 10.0), stabilizers, surfactants, preservatives, and alcohols or other polar organic solvents as well as gelling agents for the production of semi-solid media.

The media are preferably sterilized prior to use. Sterilization is preferably performed in the liquid state by filtration and/or by heat treatment (e.g. 121° C. for 15 minutes) and/or irradiation.

The pH of the dissolved medium prior to addition of cells is typically between pH 2 and 12, more preferable between pH 4 and 10, even more preferably between pH 6 and 8 and most preferable between pH 6.5 to 7.5 and ideally between pH 7.1 to 7.5.

The culture medium according to the present invention can be used under aerobic as well as anaerobic growth conditions. The person skilled in the art is familiar with the respective measures to be taken for aerobic or anaerobic growth. Typically, for anaerobic culture conditions oxygen is removed, i.e. by an additive that reduces or preferably eliminates the oxygen content of the medium. Preferably, the additive creates an anaerobic environment by preventing oxygen from intruding into the culture medium and/or by removing oxygen from the trapped air space inside the culture medium container. The additive may comprise a reducing agent, or an oxygen absorber or scavenger such as a palladium catalyst, or an enzyme, e.g. a mono- and/or di-oxygenase, and/or succinate.

It has been found that the osmoprotectant compound and the fatty acid supporting the growth of both prokaryotes and eukaryotes can be added to a broad variety of chemically defined cell culture media to improve the applicability of those media for the purpose of growing a broad range of both prokaryotes and eukaryotes.

The present invention is therefore further directed to a medium supplement comprising at least one osmoprotectant compound and at least one fatty acid. For the supplement, preferred embodiments of the osmoprotectant compound and the fatty acid are defined as described above.

The medium supplement of the present invention is typically produced and used in the same way as the cell culture media of the present invention. The supplement might be added to a dry powder medium and be thoroughly mixed with this medium prior to dissolution and use. It might also be dissolved in water or an aqueous buffer like the cell culture medium itself and mixed with the dissolved cell culture medium afterwards.

Examples of chemically defined media that can be supplemented with the medium supplement of the present invention are
a)

| Chemical | (g/L) |
|---|---|
| NaCl | 1.2 |
| K2SO4 | 1.1 |
| MgSO4•7H20 | 0.15 |
| CaCl2•6H20 | 0.02 |
| FeSO4•7H20 | 0.001 |
| CuSO4•5H20 | 0.001 |
| Na2HPO4 | 10.8 |
| KH2PO4 | 0.5 |
| (NH4)2SO4 | 10.0 |
| D-glucose | 7.0 |

(from: Appl. Microbiol. Biotechnol. (1988); 27: 474-483, Rodriguez-Aparicio et al).

b)
Media disclosed in Journal of Bacteriology, November 2007, p. 8079-8087, Palmer et al.
c)
Media disclosed in GB 2464203
d)
Media disclosed in Journal of Bacteriology, May 1980, p. 714-719, Vol. 142, No. 2, Manning and Mitchell.

The gist of the present invention is to provide a cell culture medium that is chemically defined and supports the culture/growth of a very broad range of prokaryotes and eukaryotes. Since the medium is an exact chemical formulation batch to batch production exhibits no variation and reproducibility can be guaranteed. Since the media are substantially free from animal and plant derived peptones they represent a highly reduced likelihood of being a source of contamination at their point of use such as in pharmaceutical applications or environmental testing.

The present invention is further directed to a method for culturing microorganisms in a culture medium as defined above, as well as to the use of a cell culture medium according to the present invention for the culturing of microorganisms. In a preferred embodiment the microorganisms are selected from the group consisting of bacteria, yeast and fungi.

Typically, the medium is placed in a suitable container and inoculated with the microorganisms. Suitable containers are defined above.

The temperature of incubation of the medium to allow growth of cells is typically between 0° C. and 100° C., more typically between 15° C. and 50° C., still more typically between 20 and 35° C. The samples may for example be incubated at room temperature (around 20°), e.g. at 22.5° C. or at 32.5° C.

Generally, after inoculation, the medium is incubated for a period of time to enable some growth of the microorganisms so that they can be easily detected. For traditional methods, this time can range from a minimum of less than 1 day to 14 days. Generally, the incubation time is between about 3-14 days, more generally between 7 and 14 days.

The chemically defined media according to the present invention can replace the traditional complex culture media in various applications, e.g. in the biopharmaceutical or environmental field, in food and beverage industry or in diagnostics. Exemplary applications are listed in the following:

### Sterility and Bioburden Testing:

The media according to the present invention can be used in sterility and bioburden testing. Sterility testing of in-process material and final products must be demonstrated during the manufacture of pharmaceuticals and medical devices.

A typical method for testing bioburden levels or sterility of a sample is to filter a liquid sample over a membrane filter which retains the possible contaminants of the sample and incubating the filter in the culture medium. If the sample is non-filterable, the sample can directly be inoculated into the culture medium. In the event of one or more contaminants being present, the transparent medium becomes cloudy owing to the development of the cells.

Within this regard the use of a chemically defined culture medium according to the present invention is advantageous since it is preferably a clear medium in which even a low turbidity can easily be detected.

Filters to be used in this application have a pore size small enough to trap any microorganisms in the sample. Such filters typically have a pore size from about 0.1 μm up to 1.2 μm. Preferably, the pore size is of 0.45 μm or less. The filters can be formed of any suitable material commonly used for such applications, such as regenerated cellulose, mixed cellulose esters, cellulose acetate, polyethersulfone, polyarylsulfone and polyphenylsulfone.

Holders for the filters may simply be a stainless device such as a funnel. Alternatively, disposable, presterilized and transparent filter containing devices can be used, such as the Steritest™ EZ device (Merck Millipore), which is a closed device allowing to conduct the entire test (sampling, filtration, media addition and incubation).

### Environmental Testing:

A typical method for testing for environmental levels of microbes in a facility is to filter a sample of air through a device, retaining the microorganisms on the filter. The filter can then be incubated in the culture medium according to the present invention as discussed above.

### Food and Beverage Testing:

The regular microbiological control of beverages or drinking water is also of great importance. A typical method for beverages testing is to mix 1 mL of sample to 15 mL of molten culture medium in a plate. After solidification, plates are incubated and visible colonies are counted.

### Media Fill Test:

Media fill tests are performed on a regular basis in order to verify that aseptic production processes are not affected by microbial contamination, such as spoilage bacteria, yeasts or moulds, e.g. in pharmaceutical or food and beverage industry. Typically, in a media fill test the entire process of production of a product is simulated with a sterile culture medium instead of the respective product. The medium is then filled in separate units which are subject to a sterility test as defined above.

### Pre-Enrichment Before Specific Identification:

In a further aspect the media according to the present invention can be used for pre-enrichment of microorganisms before identification. For this purpose, the sample is inoculated in the media and the microorganisms are grown for a sufficient period of time. As soon as the desired concentration is reached the sample is subjected to further treatment, such as specific microbiological identification.

A further aspect of the present invention is therefore a method of detecting microorganisms in a sample for which it is sought to determine whether it is contaminated with a living microorganism, characterized in that it comprises a step of inoculating the sample in a culture medium as defined above and a step of observing the growth of microorganisms.

The present invention is further directed to the use of a cell culture medium according to the present invention for bioburden, sterility, environmental, food and beverage or media fill testing.

The entire disclosure of all applications, patents, and publications cited above and below are hereby incorporated by reference.

### EXAMPLES

### Test Procedure for all Strains:

Filter-sterilized liquid culture media are prepared extemporaneously or stored in 125 mL glass bottles in the fridge before use.
Serial dilution of a strain solution stored in HEPES/Glycerol at −80° C. is performed in 0.9% sodium chloride solution.
Each culture medium bottle is inoculated with 20 to 50 CFU/mL.
The inoculated bottles are incubated at 22.5° C.±2.5 and 32.5° C.±2.5 for 14 days maximum depending on the strain.
Sterile bottles are incubated in parallel as control.
Microorganism development is visually observed in each bottle.

### Example 1

The growth performance of 2 microorganisms exhibiting different nutritional needs, Methylobacterium extorquens and Staphylococcus epidermidis, is assessed in a standard chemically defined medium made of amino acids ranging from 40 mg to 1 g/L, nitrogenous bases at 5 mg/L, oligo elements ranging from 10 μg/L to 1.5 mg/L, vitamins from 50 μg/L to 5.6 mg/L, dextrose, pyruvate and salts and in the same standard chemically defined medium supplemented with the combination of the osmoprotectant compound betaine 100 mg/L and the fatty acid oleic acid 20 mg/L. 100 mL of filtered-sterilized chemically defined medium are prepared extemporaneously and inoculated with less than 50 CFU/mL of microorganisms. Each inoculated culture medium bottle is incubated at 22.5° C.±2.5° C. without agitation Growth is defined by a turbidity increase monitored on a daily basis.

### Results:

| | | Standard Chemically |
|---|---|---|
| | Standard Chemically | defined medium + Oleic |
| | defined medium | acid + Betaine |
| M. extorquens | No growth | Growth |
| S. epidermidis | No growth | Growth |

### Example 2

The time growth performance of several strains is observed in the universal standard medium tryptic soy broth (TSB) and in a standard chemically defined medium which is supplemented with the combination of the osmoprotectant compound betaine 100 mg/L and the fatty acid oleic acid 20 mg/L. 100 mL of filtered-sterilized chemically defined medium is inoculated with 20 to 50 CFU/mL of microorganisms. Each inoculated culture medium bottle is incubated at 22.5° C.+/−2.5° C. (Table 1) and 32.5° C.+/−2.5° C. (Table 2) without agitation up to 14 days.

### Results:

TABLE 1
Incubation at 22.5° C. +/− 2.5° C.

| | | Visual detection in |
|---|---|---|
| | | synthetic medium |
| | Visual detection | according to the present |
| Strains | in TSB (h) | invention (h) |
| B. subtilis | 30 | 43 |
| E. coli | 24 | 26 |
| M. extorquens | 138 | 137 |
| P. aeruginosa | 45 | 45 |
| R. pickettii | 97 | 40 |
| S. aureus | 36 | 41 |
| S. epidermidis | 62 | 65 |
| P. lautus | 41 | 70 |
| M. fujisawaense | 190 | 163 |
| M. mesophilicum | 163 | 155 |

TABLE 2
Incubation at 32.5° C. +/− 2.5° C.

| | | Visual detection in |
|---|---|---|
| | | synthetic medium |
| | Visual detection | according to the present |
| Strains | in TSB (h) | invention (h) |
| B. subtilis | 18 | 18 |
| E. coli | 18 | 18 |
| M. extorquens | No growth | 184 |
| P. aeruginosa | 18 | 18 |
| R. pickettii | 42 | 42 |
| S. aureus | 18 | 24 |
| S. epidermidis | 24 | 42 |
| P. lautus | 22 | 41 |
| M. fujisawaense | 168 | 184 |
| M. mesophilicum | 168 | 192 |

The results show equivalency between TSB and the synthetic medium according to the present invention. Some water contaminants are able to grow faster in the synthetic medium.

### Example 3

In the following example the ability of the chemically defined medium to grow yeasts and molds is assessed by studying their respective visual detection time. For this purpose in each case samples of 100 ml liquid culture medium are inoculated individually with 3 yeasts and 4 molds and incubated at 22.5° C.+/−2.5° C. and 32.5° C.+/−2.5° C. without agitation.

### Results:

TABLE 3

| | Detection time at | Detection time at |
|---|---|---|
| | 22.5° C. (hours) | 32.5° C. (hours) |
| Yeast | | |
| Candida albicans ATCC ® | 42 | 18 |
| 10231 ™ | | |
| Debaryomyces hansenii DSM 3428 | 94 | 41 |
| Exophiala sp. wild type | 40 | 16 |
| Mold | | |
| Aspergillus brasiliensis ATCC ® | 42 | 24 |
| 16404 ™ | | |
| Aspergillus brasiliensis wild type | 42 | 20 |
| Aspergillus sydowii DSM 63373 | 48 | 42 |
| Penicillium commune ATCC ® | 42 | 67 |
| 10428 ™ | | |

1. Chemically defined culture medium comprising at least one osmoprotectant compound and at least one fatty acid.

2. Chemically defined culture medium according to claim 1, characterized in that the osmoprotectant compound is selected from the group consisting of betaine (trimethylglycin), ectoine, proline, choline, saccharose, trehalose and spermidine.

3. Chemically defined culture medium according to claim 1, characterized in that the osmoprotectant compound is betaine.

4. Chemically defined culture medium according to claim 1, characterized in that the concentration of the osmoprotectant compound in the medium is in the range of 10 to 360 mg/l of culture medium.

5. Chemically defined culture medium according to claim 1, characterized in that the fatty acid is selected from the group consisting of myristoleic acid, oleic acid, linoleic acid, stearic acid, palmitic acid, arachidic acid or sapienic acid.

6. Chemically defined culture medium according to claim 1, characterized in that the fatty acid is oleic acid.

7. Chemically defined culture medium according to claim 1, characterized in that the concentration of the fatty acid in the medium is in the range of 5 to 40 mg/l of culture medium.

8. Chemically defined culture medium according to claim 1, characterized in that it further comprises at least one or more saccharide components, one or more amino acids, one or more vitamins or vitamin precursors, one or more salts, one or more buffer components, one or more co-factors and one or more nucleic acid components (nitrogenous bases).

9. Chemically defined culture medium according to claim 1, further comprising at least one fluorogenic substrate.

10. A method for culturing a microorganism, comprising adding the microorganisms to the chemically defined culture medium according to claim 1.

11. A method according to claim 10, characterized in that the microorganisms are selected from the group consisting of bacteria, yeast and fungi.

12. A method according to claim 14, which is in the field of bioburden, sterility, environmental, food and beverage or media fill testing.

13. Method of preparing a culture medium according to claim 1 comprising a step of combining and mixing the components.

14. Method for detecting microorganisms in a sample for which it is sought to determine whether it is contaminated with a living microorganism, characterized in that it comprises a step of inoculating the sample in a culture medium according to claim 1 and a step of observing the growth of microorganisms.

15. Supplement for a chemically defined culture medium comprising at least one osmoprotectant compound and at least one fatty acid.
